(19) United States
(12) Patent Application Publication
(10) Pub. No.: US 2025/0129004 A1
(43) Pub. Date: Apr. 24, 2025

(54) PRODUCTION OF PHENOL FROM A BIOMASS

(71) Applicant: SYENSQO SA, Brussels (BE)

(72) Inventors: Jianxia Zheng, Shanghai (CN); Stéphane Streiff, Shanghai (CN); Sergio Mastroianni, Lyon (FR)

(73) Assignee: SYENSQO SA, Brussels (BE)

(21) Appl. No.: 18/681,467

(22) PCT Filed: Aug. 5, 2021
(86) PCT No.: PCT/CN2021/110780

§ 371 Date: Feb. 5, 2024

(51) Int. Cl. C07C 37/58; C07C 29/34; C07C 37/20; C07C 45/45; C12N 1/16

(52) U.S. Cl. CPC C07C 37/58; C07C 29/34; C07C 37/20; C07C 45/45; C12N 1/16

(57) ABSTRACT

Process for the production of phenol, said process comprising the steps of: (A) producing ethanol from a biomass, which is advantageously a carbohydrate-containing biomass, then (B) causing the ethanol produced at the step (A) to react with methanol, so as to produce benzyl alcohol and/or benzaldehyde, then (C) causing the benzyl alcohol and/or benzaldehyde produced at the step (B) to react with oxygen, so as to produce benzoic acid, then (D) causing the benzoic acid produced at the step (C) to react with oxygen, so as to produce phenol. Use of no more than 20 parts by weight of a carbohydrate contained in a biomass for the production of one part of phenol.

### TECHNICAL FIELD

The present invention relates to the production of phenol from a biomass.

### BACKGROUND ART

Phenol (C₆H₅OH) is an aromatic chemical used widely as a versatile molecule for the synthesis of various phenolic resins, bisphenol A, synthetic fibers, nylon, cyclohexanol, epoxy, pharmaceuticals, etc. Nearly all phenol is obtained by oxidizing petrochemical feedstocks like benzene and toluene, which are non-renewable and unsustainable.

Most of phenol is produced from petroleum-based benzene by cumene process, which also accounts for about 20 percent of the global benzene demand. However, this process has several disadvantages. In particular, it co-produces equimolar amounts of acetone which decreases the economic viability when acetone demand is too low. Furthermore, it involves complicated conditions, high energy consumption and causes environmental pollution.

To address this limitation, several researchers have explored various alternative routes for phenol production from renewable resources, especially from biomass. Biomass has been realized as one of the most significant sustainable substitute for petroleum-based fuels.

Pyrolysis of bio-oil is an option to produce phenol but, because of the complex composition of biomass-derived oils and thermal instability, the separation of phenol becomes costly and unfeasible from a practical standpoint.

WO 2014/076113 (to Bayer Technology Services GMBH and Bayer MaterialScience AG) provides a process for producing phenol wherein a highly sophisticated, genetically modified recombinant host strain is incubated in the presence of sugar. The recombinant host strain performs the transformation of the sugar according to a very complex sequence of reactions, as illustrated in FIG. 1 for glucose; the sequence comprises the overproduction of chorismate, its conversion to 4-hydroxybenzoate and the conversion of the 4-hydroxybenzoate into phenol. According to example 6, the recombinant host strain E. coli BW25113 ΔtyrR ΔpheAtyrA pJF119ubiChbdBCD was incubated in a shake flask or in a fermentor in the presence of a sugar-containing biomass, viz. a raw sugar cane juice with a high concentration of 1-kestose, which acted as the sole energy and carbon source. The total sugar concentration in the fermentation medium that was caused to be fermented was in each case 15 g/l. After the depletion of all sugars, the fermentation in the shake flask and in the fermentor yielded a phenol concentration of 3.1 mM and 0.9 mM respectively, corresponding to phenol yields of respectively 1.9 wt % and 0.5 wt %. Bayer's method suffers from several drawbacks. Firstly, the required genetically modified recombinant host strain has a much sophisticated structure, which makes its preparation difficult, expensive and time-consuming. Then, the reaction scheme which the strain uses for converting a sugar into phenol is much complex and essentially out of control, which results in an increased risk to achieve poorly reproducible results and in the formation of a wide variety of by-products which are much difficult to separate from the phenol. Last but not least, the yield in phenol is very poor, decreasing to even well below 1 wt % when switching from the laboratory shake flask to the fermentor.

Another possible option for the production of phenol from biomass is the use of the lignin fraction of a lignocellulosic biomass, which is the most abundant renewable source. Lignin is a high molecular weight polymer composed of methoxylated alkylphenol units. It is regarded as a rich source of phenolics. A process for the production of phenol from lignin is described by Yan J. et al., Science Advances, 2020, 6, pages: eabd1951. In this document, a zeolite catalyst is used for the direct cleavage of C-C bonds to produce hydroxyl group on the aromatic ring under mild conditions. The yield of phenol could reach about 10 wt %. This process suffers from several drawbacks, including a high catalyst loading and the formation of treatment residues during the reaction, which makes it quite expensive and results in a poor viability at industrial scale.

Finally, WO 2016/061262 (to Gevo, Inc.) describes a process for the direct conversion of bio-based ethanol to isobutylene, propylene, and/or acetone by utilizing mixed oxide catalysts or novel bi-functional heterogeneous catalysts. In one of the embodiments, it is mentioned that phenol could be produced as a co-product in very less quantity, probably well below 1 wt %. This process is primarily intended to produce functionalized lower hydrocarbons. It has consequently the drawbacks that it can only produce phenol with a very low yield and that the separation of the so-produced phenol would be much difficult among all the reaction products.

There is a need to overcome the drawbacks of the processes of the prior art. There is a need to produce phenol (C₆H₅OH) from a biomass in a reasonably high yield, viz. a yield of typically at least 5 wt %, preferably of at least 10 wt % by a process which has a good viability at industrial scale.

### SUMMARY OF THE INVENTION

These drawbacks are now overcome and these needs are now met by the present invention.

The present invention can be viewed as a process for the production of phenol, said process comprising the steps of:
(A) producing ethanol from a biomass, which is advantageously a carbohydrate-containing biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid, then
(D) causing the benzoic acid produced at the step (C) to react with oxygen, so as to produce phenol.

The present invention can also be viewed as a use of 20 parts by weight or less of a carbohydrate contained in a biomass for the production of 1 part of phenol.

These and other features, aspects and advantages of the present subject matter will be better understood with reference to the following description and appended claims.

### DETAILED DESCRIPTION

Those skilled in the art will be aware that the present disclosure is subject to variations and modifications other than those specifically described. It is to be understood that the present disclosure includes all such variations and modifications. The disclosure also includes all such steps, features, compositions and compounds referred to or indicated in this specification, individually or collectively and any and all combinations of any or more of such steps or features.

### Definitions

For convenience, before further description of the present disclosure, certain terms employed in the specification, and examples are collected here. These definitions should be read in the light of the remainder of the disclosure and understood in the same way as a person of skill in the art understands them. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The term “and/or” includes the meanings “and” and “or”.

The terms “comprise”, “include”, “comprising” and “including” are used in the inclusive, open sense, meaning that additional elements may be included. Throughout this specification, unless the context requires otherwise, these terms and variations thereof, such as “comprises”, will be understood to imply the inclusion of a stated element or step or group of element or steps but not the exclusion of any other element or step or group of element or steps. The term “including” is used to mean “including but not limited to”. “Including” and “including but not limited to” are used interchangeably.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also all the individual numerical values or sub-ranges encompassed within that range as if each numerical value and sub-range is explicitly recited. For example, a temperature range of from 100° C. to 200° C. should be interpreted to include not only the explicitly recited range of 100° C. to 200° C., but also sub-ranges, such as 110° C. to 170° C., 120° C. to 160° C., and so forth, as well as individual amounts, including fractional amounts, within the specified ranges, such as 135° C. and 145.5° C.

In a first aspect, the present invention provides a process for the production of phenol, said process comprising the steps of:
(A) producing ethanol from a biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid, then
(D) causing the benzoic acid produced at the step (C) to react with oxygen, so as to produce phenol.

According to the step (A), ethanol is produced from a biomass.

The biomass which is used in the process in accordance with the present invention is advantageously a carbohydrate-containing biomass. The carbohydrate content of the carbohydrate-containing biomass is usually of at least 2 wt %, very often at least 4 wt %, desirably at least 8 wt % and sometimes of at least 12 wt % or at least 16 wt %, based on the total weight of the carbohydrate-containing biomass. The carbohydrate-containing biomass can be natural or chemically modified. The carbohydrate contained in the carbohydrate-containing biomass can be (i) a sugar, in particular a monosaccharide (such as glucose, galactose, fructose or xylose), a disaccharide (such as sucrose, lactose or maltose) or a polyol (such as sorbitol or mannitol); (ii) an oligosaccharide such as a maltodextrin; (iii) a polysaccharide, in particular a starch (such as amylose or amylopectin) or a non-starch polysaccharide (such as glycogen, cellulose or hemicellulose); it is preferably a sugar, more preferably a monosaccharide (such as glucose, galactose, fructose or xylose). Thus, in a preferred embodiment, the biomass is a sugar-containing biomass, such as sugarcane, sugar beet, sweet sorghum and molasses; the sugar content of the sugar-containing biomass is usually of at least 2 wt %, very often at least 4 wt %, desirably at least 8 wt % and sometimes of at least 12 wt % or at least 16 wt %, based on the total weight of the sugar-containing biomass. An especially preferred biomass is a sugarcane. As herein used, the term “sugarcane” denotes both unmodified, natural sugarcane and energy canes, which are genetically modified sugarcanes to become more productive in the production of bioethanol, such as Cana-Vertix® energy cane from GranBio. In other embodiments, the biomass is a starch-containing biomass such as corn, wheat and root crops, or a lignocellulosic biomass, including crop residues (such as cane bagasse and straws of cereals), woods, herbaceous biomass, agro-industrial residues (such as sawdust and wood chips) and cellulosic wastes (such as newsprints and waste office papers).

When the biomass is a carbohydrate-containing biomass, the reaction of the step (A) consists advantageously in a fermentation of the carbohydrate contained in the carbohydrate-containing biomass. Then, the step (A) comprises preferably a separation from the carbohydrate-containing biomass, of a material comprising a higher amount of carbohydrate than the amount of carbohydrate contained in the carbohydrate-containing biomass as a whole, followed by a fermentation of said material to cause the conversion of the carbohydrate contained therein into ethanol.

As above indicated, an exemplary carbohydrate-containing biomass is sugarcane. A sugarcane comprises a stalk comprising a rind and, surrounded by the rind, a pith. The pith (also commonly referred to as sugar-fith, Comfith or derinded stalk) is a matrix of parenchyma cells in which vascular bundles are embedded. A sugarcane comprises generally sugar, especially sucrose, principally in the pith, more precisely in the parenchyma cells. These cells can be easily ruptured to free a juice comprising sugar, advantageously with a high content.

When the biomass is a sugarcane, the step (A) comprises generally the separation from the sugarcane of the pith comprising a higher amount of sugar than the amount of sugar contained in the sugarcane as a whole. Commonly employed methods to separate the pith from the sugarcane are (i) milling and/or crushing, (ii) hot water extraction and/or diffusion, or a combination of (i) and (ii). In the diffusion method, cane is prepared by knife mills and roller crusher combinations. After separation from the sugarcane, the pith comprises typically some parenchyma cells in which vascular bundles are embedded (as before the separation) and a liquid juice comprising sugar, especially sucrose, freed by the rupture of some other parenchyma cells (hereinafter, “pith juice”).

Following its separation from the carbohydrate-containing biomass, the material comprising a higher amount of carbohydrate is advantageously fermented in a reactor, generally a batch reactor, called “fermentor” to cause the conversion of the carbohydrate contained therein into ethanol. For example, following its separation from the sugarcane, the pith is advantageously fermented in a fermentor to cause the conversion of the sugar contained therein into ethanol.

To this end, a fermentation medium, which is typically in the form of a suspension, is advantageously prepared by mixing the material comprising a higher amount of carbohydrate with water. For example, a fermentation medium, which is typically in the form of a suspension, is advantageously prepared by mixing the pith with water.

The mixing can be achieved in a dedicated vessel or directly in the fermentor. The material comprising a higher amount of carbohydrate to water ratio, for example the pith to water ratio, is advantageously such that it allows for the formation of a suspension wherein the fermentation reaction proceeds efficiently. This ratio ranges generally from 0.01 to 2 w/w. Preferably, it ranges from 0.1 to 1 w/w. More preferably it ranges from 0.2 to 0.6 w/w.

The fermentation of the material comprising a higher amount of carbohydrate, for example the pith, is advantageously carried out using a microorganism. So, the fermentation medium advantageously further comprises such a microorganism, in addition to the material comprising a higher amount of carbohydrate (e.g. the pith) and the water. Preferably, the microorganism is a yeast, a bacteria or a fungus. More preferably, the microorganism is a yeast selected from the group consisting of Saccharomyces spp. and Brettanomyces custersii. Still more preferably, it is Saccharomyces cerevisiae. The most preferably, it is Saccharomyces cerevisiae CBS 2959 strain. The fermentation medium is advantageously formed by adding the microorganism to a mixture, typically a suspension, comprising water and the material comprising a higher amount of carbohydrate (e.g. the pith).

When the biomass is a carbohydrate-containing biomass, in particular a sugar, the weight amount of the microorganism, based on the total weight of the carbohydrate, ranges generally from 0.1% to 10%, preferably from 0.3% to 3%.

The fermentation of the material comprising a higher amount of carbohydrate, for example the fermentation of the pith, is advantageously carried out at an acidic pH. So, the fermentation medium comprises advantageously an acid, in addition to the material comprising a higher amount of carbohydrate, the water and the microorganism. The nature and amount of the acid is advantageously chosen in such a way that the fermentation medium has a pH from 2.5 to 5.5, preferably from 3.0 to 5.0, more preferably from 4.0 to 4.5. The nature and amount of the acid is not particularly critical provided they allow for the preparation of a fermentation medium having the desired pH. Non limitative examples of acids suitable for use in accordance with the present invention include hydrochloric acid, nitric acid, sulfuric acid, phosphoric acid, formic acid and acetic acid. The acid is preferably a strong acid, more preferably hydrochloric acid. The fermentation medium is advantageously formed by adding concomitantly the microorganism and the acid to a mixture, typically a suspension, comprising water and the material comprising a higher amount of carbohydrate (e.g. the pith).

Ethanol can be further added to the fermentation medium, generally before the fermentation takes place. The presence of a higher ethanol concentration in the fermentation medium can facilitate the conversion of the biomass.

Advantageously, the fermentation takes place at a temperature of from 10° C. to 80° C., preferably of from 15° C. to 60° C. and more preferably of from 20° C. to 40° C.

Generally, the fermentation takes place at a pressure of from 0.1 to 10 MPa, preferably from 0.5 to 5 MPa. More preferably, the reaction takes place at about the atmospheric pressure (1 atm=101.325 kPa). The reaction takes place advantageously in the presence of oxygen, preferably in an air atmosphere.

Usually, the fermentation takes place for a fermentation period of from 10 h to 100 h, preferably of from 20 h to 70 h and more preferably of from 30 h to 60 h.

As above explained, the step (A) comprises generally (A-1) causing a biomass to undergo a chemical transformation in a first reaction medium to produce ethanol, in particular causing a sugarcane pith to undergo a fermentation reaction in a fermentation medium comprising the sugarcane pith and water to produce ethanol; the fermentation medium obtained upon completion (A-1) may comprise the so-produced ethanol, the unfermented pith, the water, the microorganism and the acid.

Advantageously, following (A-1), the step (A) further comprises (A-2) separating the ethanol from the first reaction medium, in particular separating the so-produced ethanol from the fermentation medium comprising the unfermented pith, the water, the microorganism and the acid.

The so-produced ethanol can be separated from the first reaction medium using one or more methods well known to a person skilled in the art. Such methods include but are not limited to sieving, filtration, centrifugation, distillation, adsorption, solvent extraction and pervaporation. A preferred method is or includes filtration. When the biomass is sugarcane and the reaction is a fermentation reaction, the unfermented pith may comprise a fraction of solid particles which can be retained by a filter or a sieve, while the ethanol passes through the filter or the sieve, typically together with the unfermented pith juice, the water, the microorganism and the acid.

When the biomass is a carbohydrate-containing biomass and the first reaction is a fermentation, a particular embodiment of the present invention provides that part or all of the produced ethanol can be recycled, meaning that part or all of the produced ethanol can be added to a new first reaction medium comprising a biomass, and the new first reaction medium can then be caused to undergo a new chemical transformation so as to produce an additional amount of ethanol. It can be proceeded with such a recycling one or several times. As above indicated, the presence of a higher ethanol concentration in the reaction medium can facilitate the conversion of the biomass. When the biomass is sugarcane and the reaction is a fermentation reaction, the ethanol is advantageously recycled with other ingredients comprised in the fermentation medium after the reaction has taken place, such as the unfermented pith juice, the water, the microorganism and the acid.

In the step (A), when the biomass is a carbohydrate-containing biomass, the ethanol is produced in a weight amount advantageously of at least 30 wt %, preferably at least 40 wt %, more preferably at least 50 wt %, still more preferably at least 55 wt % and the most preferably at least about 60 wt %, based on the weight of the carbohydrate contained in the biomass.

According to the step (B), the ethanol produced at the step (A) is reacted with methanol to produce benzyl alcohol and/or benzaldehyde.

This reaction takes advantageously place in the presence of catalyst, which is generally supported on a support. The catalyst is preferably a transition metal catalyst, which is generally supported on a support.

The transition metal for use as the catalyst in the step (B) may be an element chosen from the elements of the d-block of the periodic table, which consists of groups 3 to 12, and the elements of the f-block of the periodic table, which consists of lanthanides and actinides. The transition metal is preferably an element of the d-block of the periodic table; it is more preferably chosen from cobalt, nickel, copper, silver, iridium, zinc and yttrium; still more preferably, it is chosen from cobalt, copper and nickel; the most preferably, it is cobalt.

The transition metal may be provided directly in metal state, with an oxidation state of 0. Alternatively, the transition metal may be embedded in a precursor, typically a metal salt such as a nitrate, a chloride, a levulinate, a sulfate or an acetate salt, wherein the oxidation state of the transition metal is above 0, possibly +1, +2, +3 or +4, preferably +2.

The catalyst, in particular the transition metal catalyst, may be unsupported. Alternatively and preferably, the catalyst, in particular the transition metal catalyst, is supported on a support. As suitable supports of the catalyst, it can be notably cited fumed or colloidal silica, ceramics, metal aluminates, metal silicates, metal aluminosilicates (such as zeolites), metal oxides, metal hydroxides, wherein the metal can be, for example, an alkaline earth metal or a transition metal such as lead. It can also be cited metal (hydrogeno) phosphates, metal hydroxy (hydrogeno) phosphates (such as hydroxyapatites) and metal halogenophosphates (such as fluoroapatites and chloroapatites), wherein the brackets surrounding “hydrogeno” denote that the aforesaid metal compounds can include one or more phosphates and/or one or more hydrogenophosphates groups (HPO₄⁻) and wherein the metal can be, for example, an alkaline earth metal or a transition metal such as lead. In a preferred embodiment, the support is an apatite, especially an apatite chosen from hydroxyapatites, chloroapatites and fluoroapatites; the apatite, especially the hydroxyapatite, the chloroapatite or the fluoroapatite, can be a stoichiometric or non-stoichiometric compound. In another preferred embodiment, the support is a stoichiometric compound of chemical formula MgₓCa_{y}Sr_{z}BaₘPbₙ(OH)ₐCl_{b}F_{c}(PO₄)_{d} wherein x, y, z, m, n, a, b and c are integers greater than or equal to 0, d is an integer greater than 0, x+y+z+m+n is greater than 0, a+b+c is greater than 0 and wherein 2x+2y+2z+2m+2n-a-b-c-3d=0, especially a stoichiometric compound of chemical formula Ca_{y}(OH)ₐCl_{b}F_{c}(PO₄)_{d} wherein y and d are integers greater than 0, a, b and c are integers greater than or equal to 0, a+b+c is greater than 0 and wherein 2y-a-b-c-3d=0. More preferably, the support is a hydroxyapatite of formula Ca₁₀(OH)₂(PO₄)₆. Good results are obtained when the catalyst of the step (B) is cobalt supported on a support which is a hydroxyapatite of formula Ca₁₀(OH)₂(PO₄)₆, commonly referred to as a Co/HAP catalyst.

When the catalyst, in particular the transition metal catalyst, is supported on a support, its loading is generally from 0.1 wt % to 2 wt %, preferably from 0.5 wt % to 1.5 wt %, based on the weight of the support.

Before its use at the step (B), the catalyst is advantageously pre-treated in a hydrogen/inert gas mixed atmosphere, generally at a temperature of from 300° C. to 700° C., preferably from 350° C. to 450° C. The inert gas is generally nitrogen. The concentration of the hydrogen atmosphere is advantageously of from 3% to 15% H₂/inert gas. Preferably, the concentration of the hydrogen atmosphere is of from 5% to 10% of H₂/inert gas.

The step (B) comprises generally (B-1) causing the ethanol which has been separated from the first reaction medium to react with methanol in a second reaction medium. The reaction of the step (B) can take place in a batch or continuous reactor. Preferably, the reactor is a continuous reactor, more preferably a packed-bed reactor. When the reaction takes place in a continuous reactor, in particular in a packed-bed reactor, the ethanol and the methanol are continuously fed in the reactor.

In general, the reaction takes place in the second reaction medium at a pressure ranging from 0.1 to 10 MPa, preferably from 0.5 to 5 MPa. Preferably, the reaction takes place at about the atmospheric pressure (1 atm=101.325 kPa). The reaction takes place advantageously in an inert atmosphere, preferably in a nitrogen atmosphere. The total partial pressure of ethanol and methanol in the second reaction medium usually ranges from 1 to 20 kPa, preferably from 3 to 12 kPa and more preferably from 4 to 8 kPa.

The initial methanol to ethanol weight ratio, viz. the weight ratio before the methanol and the ethanol are reacted together, usually ranges from 0.5 to 5, often from 0.8 to 3. It is preferably of at least 1, more preferably of at least 1.1 and still more preferably of at least 1.2. Besides, it is preferably of at most 2 and more preferably of at most 1.5. When the second reaction takes place in a reactor wherein the ethanol and the methanol are continuously fed, the molar ratio of ethanol and methanol can be adjusted by controlling the flow rate of the ethanol and methanol.

At the step (B), methyl benzaldehyde and/or methyl benzyl alcohol can be formed as by-products; by co-feeding ethanol and methanol with an appropriate methanol to ethanol weight ratio as above detailed, a higher selectivity towards the benzyl alcohol and/or benzaldehyde can be achieved. In contrast, if the ethanol were fed alone, methyl benzaldehyde and/or methyl benzyl alcohol would be formed as the only aromatic products.

In general, the reaction of the step (B) takes place at a temperature of from 200° C. to 500° C. Preferably, the temperature of the reaction ranges from 300° C. to 400° C. More preferably, it ranges from 325° C. to 375° C.

The catalytic conversion takes place for a time period (in case of a batch reaction) or residence time (in case of a continuous process) which usually ranges from 0.01 h to 100 h, and often from 0.1 h to 10 h.

The weight amount of ethanol which is converted during the step (B) is generally of at least 10 wt %, preferably at least 15%, and may be of at least 30%, at least 45% or at least 60%, at least 75% or at least 90%. Indeed, any unconverted ethanol, after separation of the reaction products as will be detailed later on, can be re-used as reactant of a new second reaction medium, and the unconverted ethanol can be recycled as many times as desired, possibly until it is essentially fully converted.

In general, both benzyl alcohol and benzaldehyde are produced in the step (B). The weight amount of the benzyl alcohol, based on the combined weight amount of the benzyl alcohol and the ethanol, may vary to some extent depending notably on the temperature at which the ethanol and the methanol are reacted; it ranges generally from 50 wt % up to less than 100 wt %, very often from 70 wt % to 95 wt %, possibly from 85 wt % to 95 wt %.

Advantageously, following the sub-step (B-1), the step (B) further comprises (B-2) separating the benzyl alcohol and/or benzaldehyde from the second reaction medium; more specifically, the sub-step (B-2) allows typically for the separation of benzyl alcohol and/or benzaldehyde from the unconverted ethanol, the unconverted methanol and unwanted reaction by-products, in particular 2-methyl benzyl alcohol, 4-methylbenzyl alcohol, 2-tolualdehyde and 4-tolualdehyde. The sub-step (B-2) can be achieved by using one or more methods well known to a person skilled in the art. A preferred method is or includes distillation. The distillation is advantageously operated using a single distillation column. The distillation column may operate at a temperature of from 100° C. to 200° C., preferably at a temperature of from 110° C. to 170° C., more preferably at a temperature of from 120° C. to 160° C. The distillation column may operate under vacuum.

Preferably, the sub-step (B-2) provides further that the unconverted ethanol is separated from the second reaction medium and that the so-separated unconverted ethanol is recycled, that is to say that it is re-used as reactant of a new second reaction medium in accordance with the sub-step (B-1); the unconverted ethanol can be recycled as many times as desired, possibly until it is fully or essentially fully converted. Also preferably, the sub-step (B-2) provides further that the unconverted methanol is separated from the second reaction medium and that the so-separated unconverted methanol is recycled, that is to say that it is re-used as reactant of a new second reaction medium in accordance with the sub-step (B-1); the unconverted methanol can be recycled as many times as desired, which can be one or several times, possibly until it is fully or essentially fully converted. Very preferably, the sub-step (B-2) provides further that the unconverted ethanol and the unconverted methanol are separated from the second reaction medium and that the so-separated unconverted ethanol and methanol are recycled, that is to say that they are re-used as reactants of a new second reaction medium; the unconverted ethanol and the unconverted methanol can be recycled as many times as desired, possibly until they are fully or essentially fully converted.

In the step (B), in the absence of any unconverted ethanol recycling, the benzyl alcohol and/or the benzaldehyde are produced in a combined weight amount, viz. the weight of the benzyl alcohol plus the weight of the benzaldehyde, of advantageously of at least 10 wt %, possibly at least 15 wt % or at least 20 wt %, based on the weight of the ethanol which is used as reagent.

In the step (B), the selectivity in the benzyl alcohol and/or the benzaldehyde can exceed 20%, 25%, 30% or even 35%. So, when the unconverted ethanol is recycled and further reacted, desirably until it is fully or essentially fully converted, the benzyl alcohol and/or the benzaldehyde can be produced in a combined weight amount of at least 20 wt %, possibly at least 25 wt %, at least 30 wt % or at least 35 wt %, based on the initial weight of the ethanol.

According to the step (C), the benzyl alcohol and/or the benzaldehyde produced at the step (B) are caused to react with oxygen, so as to produce benzoic acid.

The reaction of the step (C) is typically an oxidation reaction.

The oxygen which is reacted during the step (C) may be provided as such (that is to say unmixed with other gases such as nitrogen and rare gases) or in admixture with one or more inert gases. The reaction of the step (C) is advantageously performed in an air atmosphere, wherein the air comprises oxygen to be caused to react with the benzyl alcohol and/or the benzaldehyde. The case being, the reaction of the step (C) is performed at an air pressure which is advantageously in the range of 0.1 to 10 bar, preferably from 0.5 to 2 bar. More preferably, the reaction of the step (C) is conducted at about the atmospheric pressure (1 atm=101.325 kPa).

The reaction of the step (C) is advantageously performed in the presence of a catalyst, which is generally supported on a support. Preferably, the reaction of the step (C) is advantageously performed in the presence of a catalyst and of a co-catalyst, which are generally supported on a support.

The catalyst is preferably a transition metal catalyst, which is generally supported on a support.

The transition metal for use as catalyst in the step (C) may be an element chosen from the elements of the d-block of the periodic table, which consists of groups 3 to 12, and the elements of the f-block of the periodic table, which consists of lanthanides and actinides. Its electronegativity is advantageously of at least 1.9, as it is the case for noble metals and also for silver (Ag), rhenium (Re), copper (Cu) and mercury (Hg). Preferably, the transition metal is a noble metal. As herein used, the term “noble metal” denotes any element chosen from ruthenium (Ru), rhodium (Rh), palladium (Pd), osmium (Os), iridium (Ir), platinum (Pt) and gold (Ag). More preferably, the transition metal is chosen from ruthenium, rhodium, palladium, iridium and platinum. Still more preferably, the transition metal is platinum.

The co-catalyst is preferably a poor metal catalyst, which is generally supported on a support. Also preferably, the co-catalyst is supported on the same support as the catalyst.

The poor metal for use as catalyst in the step (C) is to be found among the elements of group 12 to 16 of the periodic table of the elements, with the proviso that it differs from an element of group 12 when the transition metal for use as catalyst in the step (C) is an element of such group 12. As herein used, the term “poor metal” denotes any element chosen from zinc (Zn), cadmium (Cd), mercury (Hg), aluminum (Al), gallium (Ga), indium (In), thallium (Tl), germanium (Ge), tin (Sn), lead (Pb), antimony (Sb), bismuth (Bi), tellurium (Te) and polonium (Po). Among them, aluminum, gallium, indium, thallium, tin, lead, bismuth and polonium are commonly referred to as “post-transition metals”. The poor metal is preferably chosen from aluminum, gallium, indium, thallium, germanium, tin, lead, antimony, bismuth and polonium. More preferably, the poor metal is chosen from tin, lead, antimony, bismuth and polonium. Still more preferably, the poor metal is bismuth.

The catalyst (in particular, the transition metal catalyst) and, when present, the co-catalyst (in particular, the poor metal co-catalyst) may be unsupported. Alternatively and preferably, the catalyst (in particular, the transition metal catalyst) and, when present, the co-catalyst (in particular, the poor metal co-catalyst) are supported on a support. As suitable supports of the catalyst and, when present, the co-catalyst, it can be notably cited zeolites, Kieselguhr, silica, alumina, silica-alumina, clay, titania, zirconia, magnesia, calcia, lanthanum oxide, niobium oxide and activated carbon. Preferably, the support is chosen from the group consisting of activated carbon, alumina, clay and titania. More preferably, the support of the catalyst and, when present, the co-catalyst is activated carbon.

Good results are obtained when the catalytic system of the step (C) is a platinum catalyst combined with a bismuth co-catalyst, both being supported on a support which is activated carbon. This catalytic system is commonly referred to as a Pt-Bi/C catalyst.

The weight amount of the catalyst (in particular, the transition metal catalyst) which is advantageously used in the reaction of the step (C) typically from 1 wt % to 10 wt %, preferably from 3 wt % to 7 wt %, more preferably from 4 wt % to 6 wt %, based on the combined weight of the benzyl alcohol and the benzaldehyde.

The weight amount of the co-catalyst (in particular, the poor metal catalyst) which is preferably used in combination with the catalyst in the reaction of the step (C) ranges typically from 10 wt % to 100 wt %, preferably from 15 wt % to 70 wt %, more preferably from 20 wt % to 50 wt %, based on the weight of the catalyst.

When the catalyst, in particular the transition metal catalyst, is supported on a support, its loading is generally from 0.05 wt % to 10 wt %, preferably from 0.1 wt % to 2 wt %, based on the weight of the support.

The reaction of the step (C) is advantageously performed in the presence of a base, such as K₂CO₃, NH₃ or a strong base. The base is preferably a strong base, such as Ca(OH)₂, Al(OH)₃ or an alkali metal hydroxide. The base is more preferably an alkali metal hydroxide, such as KOH, NaOH or LiOH. Still more preferably, the base is KOH.

The weight amount of the base used in the reaction ranges generally from 10 wt % to 1000 wt %, preferably from 100 wt % to 500 wt %, based on the combined weight of the benzyl alcohol and/or the benzaldehyde.

The reaction of the step (C) is advantageously performed in the presence of a solvent. The solvent used in the reaction of the step (C) is typically water, a C₁-C₁₂ aliphatic alcohol or a mixture thereof. It is preferably water, a C₁-C₄ alkanol or a mixture thereof. As the C₁-C₄ alkanol, methanol and ethanol are preferred. More preferably, the solvent is a mixture of methanol and water.

Advantageously, the reaction of the step (C) takes place at a temperature of from 10° C. to 100° C., preferably of from 30° C. to 80° C., more preferably of from 40° C. to 60° C.

Usually, the reaction of the step (C) takes place for a time period of from 30 min to 30 h, preferably from 1 to 10 h. The time period during which the reaction of the step (C) takes place allows advantageously for reaching a conversion into benzoic acid of at least 80 wt %, preferably at least 90 wt %, more preferably at least 95 wt %, based on the combined weight of the benzyl alcohol and/or the benzaldehyde.

As above detailed, the step (C) comprises generally (C-1) causing the benzyl alcohol and/or the benzaldehyde which have been separated from the second reaction medium to react with oxygen in a third reaction medium, so as to produce benzoic acid in the third reaction medium.

The resulting third reaction medium is advantageously acidified using an acid. Non limitative examples of acids suitable for acidifying the resulting reaction medium include hydrochloric acid, nitric acid, sulfuric acid, phosphoric acid, formic acid and acetic acid. The acid is preferably a strong acid, more preferably hydrochloric acid.

After the acidification, the step (C) comprises desirably (C-2) separating the benzoic acid from the third reaction medium. The so-produced benzoic acid can be separated from the third reaction medium using one or more methods well known to a person skilled in the art. Such methods include but are not limited to filtration, centrifugation, distillation, adsorption, solvent extraction and pervaporation. Usually, the catalyst is firstly separated from the resulting reaction mixture, preferably by centrifugation. The remaining liquid part is then advantageously extracted with a solvent, preferably an ester, more preferably an alkyl alkanoate with ethyl acetate being especially recommended. Thereafter, the benzoic acid is advantageously separated from the so-extracted liquid, using preferably a distillation column under vacuum.

The temperature in the distillation column may range from 125° C. to 250° C., possibly from 150° C. to 225° C. or from 170° C. to 200° C. Alternatively, the benzoic acid may be separated directly from the remaining liquid part (without further solvent extraction) or directly from the raw third reaction medium resulting from (C-1).

In the step (C), the benzoic acid is produced in a weight amount of advantageously at least 80 wt %, preferably at least 90 wt % and more preferably at least 95 wt %, based on the combined weight of the benzyl alcohol and the benzaldehyde.

According to the step (D), the benzoic acid produced at the step (C) is caused to react with oxygen, so as to produce phenol.

The reaction of the step (D) is typically an oxidation reaction.

The oxygen which is reacted during the step (D) may be provided as such (that is to say unmixed with other gases) or in admixture with one or more gases. It is advantageously provided as an air component, i.e. the reaction of the step (D) is advantageously performed in an air atmosphere, wherein the air comprises the oxygen to be caused to react with the benzoic acid. The case being, the air to benzoic acid weight ratio ranges generally from 0.5 to 100, preferably from 1 to 30 and more preferably from 2 to 10.

The reaction of the step (D) takes advantageously place in the presence of a catalyst.

Advantageously, the catalyst comprises, consists essentially of or consists of an oxide of a transition metal. The transition metal forming the oxide of the catalyst in the step (D) may be an element of the d-block and/or an element of the f-block of the periodic table. The transition metal is preferably an element of the d-block of the periodic table. More preferably, it is an element chosen from the elements of groups 8 to 10 of the periodic table (hereinafter, the “first element”), optionally in combination with an element chosen from the elements of groups 4 to 7 (hereinafter, the “other element”).

As examples of the first element, it can be cited iron (Fe), ruthenium (Ru), osmium (Os), cobalt (Co), rhodium (Rh), iridium (Ir), nickel (Ni), palladium (Pd), platinum (Pt) and mixtures thereof. Among them, special mention can be made, notably for cost reasons, to three of them which are non-noble metals, viz. iron (exemplary oxides thereof being FeO, Fe₂O₃ and Fe₃O₄), cobalt (exemplary oxides thereof being CoO, Co₂O₃ and Co₃O₄) and nickel (an exemplary oxide thereof being NiO). In terms of catalytic performance, the first element is preferably a mixture of an element of group 8 and an element of group 10; more preferably, it is a mixture of iron and nickel, so the corresponding oxide is typically a mixture of FeO and/or Fe₂O₃ on the one hand and NiO on the other hand, especially a mixture of Fe₂O₃ and NiO. When the catalyst comprises, consists essentially of or consists of a mixture of Fe₂O₃ and NiO, the ratio of NiO to Fe₂O₃ in the catalyst is generally from 0.001 w/w to 5 w/w, preferably from 0.1 w/w to 1.5 w/w; when the ratio exceeds 5 w/w, the amount of CO and CO₂ by-products generated by complete combustion increases, and the selectivity to phenol decreases.

As examples of the other element, it can be cited titanium (Ti), zirconium (Zr), hafnium (Hf), vanadium (V), niobium (Nb), tantalum (Ta), chromium (Cr), molybdenum (Mo), tungsten (W), manganese (Mn), technetium (Tc), rhenium (Re) and mixtures thereof. The other element is preferably chosen from titanium (exemplary oxides thereof being TiO, Ti₂O₃ and TiO₂), zirconium (an exemplary oxide thereof being ZrO₂), vanadium (exemplary oxides thereof being VO, V₂O₃, VO₂ and V₂O₅), chromium (exemplary oxides thereof being CrO, Cr₂O₃, CrO₂ and CrO₃), molybdenum (exemplary oxides thereof being MoO₂ and MoO₃) and manganese (exemplary oxides thereof being MnO, Mn₃O₄, Mn₂O₃, MnO₂, MnO₃ and Mn₂O₇); more preferably, it is vanadium and/or molybdenum; still more preferably, it is vanadium, and the corresponding oxide is typically chosen from VO, V₂O₃, VO₂ and V₂O₅, especially V₂O₅. The ratio of the oxide of the other element to the oxide of the first element in the catalyst ranges generally from 0 w/w to 1 w/w, preferably from 0.001 to 0.30 w/w and. more preferably from 0.01 to 0.10 w/w. By choosing an appropriate ratio of the oxide of the other element to the oxide of the first element in the catalyst as previously detailed, the formation of carbon monoxide and carbon dioxide by-products generated by the complete combustion can be minimized.

Good results are obtained when the catalyst comprises, consists essentially of or consists of a mixture of Fe₂O₃ and NiO. Very good results are obtained when the catalyst comprises, consists essentially of or consist of a mixture of Fe₂O₃, NiO and V₂O₅.

Preferably, the catalyst further comprises, in addition to the oxide of the transition metal, an oxide of an alkali or alkaline earth metal. More preferably, the catalyst consists essentially of or consists of an oxide of a transition metal and an oxide of an alkali or alkaline earth metal. The conversion of the benzoic acid to the phenol can be improved by the incorporation of such an oxide of an alkali or alkaline earth metal to the oxide of the transition metal; in particular, less carbon monoxide and less carbon dioxide by-products can be produced.

The alkali or alkaline earth metal has preferably an electronegativity of at most 1.0; besides, it is preferably an alkali metal. More preferably, the alkali or alkaline earth metal is chosen from lithium, sodium, potassium and rubidium. Still more preferably, it is sodium and the corresponding oxide is Na₂O.

Excellent results are obtained when the catalyst consists essentially of or consists of a mixture of Fe₂O₃, NiO, V₂O₅ and an alkali metal chosen from Li₂O, Na₂O, K₂O, Rb₂O and mixtures thereof, in particular when the catalyst consists essentially of or consists of a mixture of Fe₂O₃, NiO, V₂O₅ and Na₂O.

The ratio of the oxide of the alkali or alkaline earth metal to the oxide of the transition metal in the catalyst ranges generally from 0 w/w to 1 w/w, preferably from 0.001 to 0.100 w/w and. more preferably from 0.003 to 0.030 w/w.

The weight amount of the catalyst used in the step (D), based on the weight of the benzoic acid, ranges generally from 0.1 wt ‰ to 10 wt %, preferably from 0.3 wt ‰ to 3 wt % and more preferably from 1.0 wt ‰ to 10 wt ‰. The catalyst can be prepared using a method well known to the skilled person, including calcination, precipitation, kneading or impregnation. An exemplary preparation of a NiO—Fe₂O₃—Na₂O—V₂O₅ catalyst is provided in the examples section of the present specification.

The reaction of the step (D) takes advantageously place in the presence of water. Like the oxygen, the water can be caused to oxidize the benzoic acid. To this end, the water is advantageously provided in its vapor phase, as steam. In a preferred embodiment, the reaction of the step (D) is advantageously performed in an atmosphere comprising air and steam. The case being, the steam to benzoic acid weight ratio ranges generally from 1 to 500, preferably from 5 to 100 and more preferably from 10 to 50.

The temperature at which the reaction of the step (D) takes place is generally greater than 100° C., preferably from 150° C. to 600° C. and more preferably from 200° C. to 500° C.

The reaction pressure is advantageously any pressure capable of keeping the benzoic acid, the oxygen and, when present, the water in a gaseous phase under the reaction conditions. It may be the atmospheric pressure.

The step (D) comprises generally (D-1) causing the benzoic acid which has been separated from the third reaction medium to react with oxygen in a fourth reaction medium. The reaction of the step (D) can take place in a batch or continuous reactor. Preferably, the reactor is a continuous reactor, more preferably a fixed-bed or a fluidized-bed reactor. When the reaction takes place in a continuous reactor, in particular in a fixed-bed or fluidized-bed reactor, the benzoic acid and the oxygen are continuously fed in the reactor.

In a preferred embodiment, the benzoic acid, air and steam are continuously fed in the reactor. The space velocity of the benzoic acid, the air and the steam in the reactor is generally from 100 h⁻¹ to 8000 h⁻¹, preferably it is from 500 h⁻¹ to 4000 h⁻¹ and more preferably from 1000 h⁻¹ to 2000 h⁻¹. When the space velocity is less than 100 h⁻¹, the space time yield is insufficient. On the other hand, when the space velocity is beyond 8000 h⁻¹, the conversion of the benzoic acid to the phenol is low.

Advantageously, following (D-1), the step (D) further comprises (D-2) separating the phenol from the fourth reaction medium; more specifically, (D-2) allows typically for the separation of the phenol from the unconverted benzoic acid and possible reaction by-products. (D-2) can be achieved by using one or more methods well known to a person skilled in the art. Following (D-1), the fourth reaction medium can be rinsed with a solvent, in particular a ketone like acetone. A preferred method used in accordance with (D-2) is or includes distillation. The distillation column may operate at a temperature of from 50° C. to 200° C., preferably at a temperature of from 80° C. to 150° C., more preferably at a temperature of from 100° C. to 130° C. The distillation column may operate under vacuum.

In the step (D), the phenol can be produced with a selectivity exceeding 90%. In the step (D), the phenol can be produced in a weight amount of at least 40 wt %, preferably at least 50 wt % and more preferably at least 55 wt %, based on the weight of the benzoic acid.

Summarizing all the above, the steps (A), (B), (C) and (D) of the process in accordance with the present invention can be characterized in that:
the step (A) comprises (A-1) causing a biomass to undergo a chemical transformation in a first reaction medium, so as to produce ethanol in the first reaction medium, then (A-2) separating the ethanol from the first reaction medium;
the step (B) comprises (B-1) causing the so-separated ethanol to react with methanol in a second reaction medium, so as to produce benzyl alcohol and/or benzaldehyde in the second reaction medium, then (B-2) separating the benzyl alcohol and/or benzaldehyde from the second reaction medium;
the step (C) comprises (C-1) causing the so-separated benzyl alcohol and/or benzaldehyde to react with oxygen in a third reaction medium, so as to produce benzoic acid in the third reaction medium, then (C-2) separating the benzoic acid from the third reaction medium; and
the step (D) comprises (D-1) causing the so-separated benzoic acid to react with oxygen in a fourth reaction medium, so as to produce phenol in the fourth reaction medium, then (D-2) separating the phenol from the fourth reaction medium.

The present invention can also be viewed as a use of 20 parts by weight or less of a carbohydrate contained in a biomass for the production of 1 part of phenol. Preferably at most 12, more preferably at most 10 and still more preferably at most 8 parts by weight of the carbohydrate contained in the biomass are used for producing 1 part of phenol. For the avoidance of doubt, the use in accordance with the present invention does neither include the embodiment wherein a carbohydrate-free biomass is used for the production of phenol, nor the embodiment wherein no carbohydrate of a carbohydrate-containing biomass is used for the production of phenol; instead, in accordance with the invented use, usually at least 1, often at least 2, sometimes at least 4, possibly at least 5 or at least 6 parts by weight of a carbohydrate contained in a biomass need to be used for producing 1 part of phenol. The carbohydrate-containing biomass involved in the use in accordance with the present invention is the same as the carbohydrate-containing biomass involved in the process in accordance with the present invention and complies advantageously with any one of the features here above described in connection with the carbohydrate-containing biomass involved in the process in accordance with the present invention.

The phenol produced by the process according to the present invention can be used as such as an antiseptic or as a local anesthetic. It can also be used for the chemical matrixectomy for ingrown finger or toenails.

The phenol produced by the process according to the present invention can also be used for the synthesis of a phenol derivative.

As a first example, it can be used for the synthesis of a phenolic resin such as polyoxybenzylmethylenglycolanhydride, better known as Bakelite® resin; to synthesize polyoxybenzylmethylenglycolanhydride, the phenol produced by the process according to the present invention is generally condensed with formaldehyde.

As another example, the phenol produced by the process according to the present invention can be used for the manufacture of a bisphenol compound. Accordingly, the present invention also concerns a method for the synthesis of a bisphenol compound, said method comprising:
producing phenol by the process as above described, then
causing the so-produced phenol to be condensed with an aldehyde or a ketone, or to be sulfonated and condensed with sulfuric acid or sulfur trioxide.

The phenol may be condensed with an aldehyde. Exemplary aldehydes suitable for use in the invented method are formaldehyde and acetaldehyde. When the aldehyde is formaldehyde, bisphenol F is synthesized. When the aldehyde is acetaldehyde, bisphenol E is synthesized.

The phenol may be condensed with a ketone. Exemplary ketones for use in accordance with the invented method are acetone, hexafluoroacetone, methyl ethyl ketone, acetophenone, benzophenone, cyclohexanone and 3,3,5-trimethylcyclohexanone. So:
when the ketone is acetone, bisphenol A is synthesized;
when the ketone is hexafluoroacetone, bisphenol AF is synthesized;
when the ketone is methyl ethyl ketone, bisphenol B is synthesized;
when the ketone is acetophenone, bisphenol AP is synthesized;
when the ketone is benzophenone, bisphenol BP is synthesized;
when the ketone is cyclohexanone, bisphenol Z is synthesized; and
when the ketone is 3,3,5-trimethylcyclohexanone, bisphenol TMC is synthesized.

The condensation reaction takes advantageously place in the presence of a strong acid, such as hydrochloric acid or a sulfonated polystyrene resin. The reaction proceeds typically as shown below for the synthesis of bisphenol A:

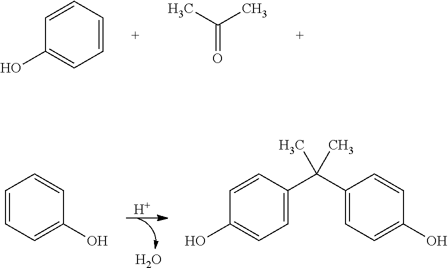

A large excess of phenol may be used to ensure full condensation.

The phenol may also be sulfonated and condensed (dehydrated) with sulfuric acid or sulfur trioxide. Then, bisphenol S is synthesized; the reaction which takes place is typically

2 C₆H₅OH+H₂SO₄→(C₆H₄OH)₂SO₂+2H₂O

or

2 C₆H₅OH+SO₃→(C₆H₄OH)₂SO₂+H₂O,

depending on whether sulfuric acid or sulfur trioxide is used as sulfonation and condensation (dehydration) agent.

The process for the production of phenol in accordance with the present invention overcomes the drawbacks of the prior art processes and has numerous advantages. It provides a new sustainable way of producing phenol from a renewable biomass. It uses advantageously low amounts of catalysts, especially when compared to the academic process using zeolite catalyst, which has been proposed by Yan in Science Advances, 2020, 6, pages: eabd1951. The phenol produced by the process of the present invention can be easily separated from its reaction medium, and the same holds true for all the intermediates produced at the different steps of the invented process, viz. the ethanol, the benzyl alcohol and/or the benzaldehyde and the benzoic acid. The phenol can be obtained with a reasonably high yield, which can be well above 5 wt % and which can even substantially exceed 10 wt % when the unconverted ethanol is recycled at the step (B) of the invented process (as herein used, the yield is the ratio of the weight of the produced phenol to the weight of the useful reagent fraction contained in the biomass, typically the carbohydrate fraction). All in all, it is contemplated that the presently invented process, is able to combine outstandingly the “green” attribute, viz. the use of a renewable biomass as starting reagent, with a good viability at industrial scale and a reasonably high economic attractiveness.

Should the disclosure of any patents, patent applications, and publications which are incorporated herein by reference conflict with the description of the present application to the extent that it may render a term unclear, the present description shall take precedence.

### EXAMPLES (IN ACCORDANCE WITH THE INVENTION)

### Materials

Ca(NO₃)_{2·}4H₂O, CAS No. 13477-34-4, Sinopharm; (NH₄)₂HPO₄, CAS No. 7783-28-0, Sinopharm; NH₃·H₂O, CAS No. 7664-41-7, 25 wt % in H₂O, Sinopharm; Co (NO₃)₂·6H₂O, CAS No. 10026-22-9, Sinopharm; Ni(NO₃)₂·6H₂O, CAS No. 13478-00-7, Sinopharm; Fe(NO₃)₃·6H₂O, CAS No. 7782-61-8, Sinopharm; NaOH, CAS No. 1310-73-2, Sinopharm; Na₂CO₃, CAS No. 497-19-8, Sinopharm; NH₄VO₃, CAS No. 7803-55-6, Sinopharm; Bio-ethanol, CAS No. 64-17-5, CREMER OLEO GmbH & Co. KG; Bio-methanol, CAS No. 67-56-1, Merck; Benzaldehyde (BA=O), CAS No. 100-52-7, J&K; Benzyl alcohol (BA-OH), CAS No. 100-51-6, J&K; 2-Methylbenzyl alcohol (MB-OH), CAS No. 89-95-2, J&K; 4-Methylbenzyl alcohol (MB-OH), CAS No. 589-18-4, J&K; Propanol, CAS No. 71-23-8, J&K; Butanol, CAS No. 71-36-3, J&K; Pentanol, CAS No. 71-41-0, J&K; Hexanol, CAS No. 111-27-3, J&K; Acetaldehyde, CAS No. 75-07-0, J&K; E-2-butenal, CAS No. 123-73-9, J&K; 2-Tolualdehyde (2-MB=O), CAS No. 529-20-4, J&K; 4-Tolualdehyde (4-MB=O), CAS No. 104-87-0, J&K; 5 wt % Pt-1.5 wt % Bi/C (moisture: 46.8%), Type 160, CAS No. 7440-06-4, Johnson Matthey; KOH, CAS No. 1310-58-3, 95% purity, Sinopharm; HCl, CAS No. 7647-01-0, 37 wt %, Sinopharm; Ethyl acetate, CAS No. 141-78-6, Sinopharm.

### Synthesis of HAP

Hydroxyapatite (HAP) is synthesized by a precipitation method. An aqueous solution of Ca(NO₃)₂·4H₂O (0.6 M, Sinopharm) is added dropwise to a solution of (NH4)₂HPO₄ (0.4 M, Sinopharm). Then, NH₃·H₂O (25 wt % in H₂O, Sinopharm) is added to the solution to achieve an initial system having a pH of 10.3. The slurry is stirred for 24 h at 80° C. After filtration, drying, and calcination (600° C. for 2 h in static air), a white HAP is obtained. The overall Ca/P ratio is about 1.67, as measured using an inductively-coupled plasma device (ICP).

### Synthesis of Co/HAP catalyst

HAP is impregnated with an 0.35 M aqueous solution of Co(NO₃)₂ ·6H₂O by incipient wetness impregnation. The slurry is stirred at room temperature for 30 min. After drying at 50° C. for 12 h in air and calcination at 350° C. for 2 h in air, the as-prepared Co/HAP catalyst is obtained. The actual Co content is about 0.8 wt %, as determined by ICP, and the chemical state of Co is +2, as determined by XPS.

### Synthesis of NiO—Fe₂O₃—Na₂O—V₂O₅ Catalyst

NiO—Fe₂O₃complex oxide catalyst is prepared by co-precipitation using an aqueous solution of Ni(NO₃)₂·6H₂O, Fe(NO₃)₃·6H₂O and NaOH. 144 g of Ni(NO₃)₂·6H₂O and 101 g of Fe(NO₃)₃·6H₂) are dissolved in 400 mL of pure water, and about 100 g of NaOH are dissolved in 500 mL of pure water. Both solutions are added dropwise to 2 L of pure water at room temperature so as to maintain the system at a pH in the range of 7-8. After adding the solutions, the mixture is stirred at room temperature for about 1 h. The precipitates obtained are washed with pure water until they become free from sodium anion, then dried at 110°° C. for 24 h in air. After calcination at 800° C. for 3 h in air, the catalysts are pulverized to particles in the range of 20-40 mesh.

Modifications with Na₂O and V₂O₅ are performed using an impregnation method with an aqueous solution of Na₂CO₃ and NH₄HO₃. 100 mL of an aqueous solution containing 1.37 g of Na₂CO₃ and 1.55 g of NH₄VO₃ are added to the above NiO—Fe₂O₃ complex oxide catalyst, and stirred at room temperature for about 1 h. The precipitates obtained are filtered and dried in the atmosphere at 120° C. for 24 h, then calcined in the atmosphere at 800° C. for 4 h to obtain a catalyst having a weight ratio of about 46:50:1:3 as NiO—Fe₂O₃—Na₂O—V₂O₅.

### Fermentation of Sugar Cane to Ethanol, in Accordance With Step (A) of the Invented Process

Freshly cut cane stalks are milled in a cane separator and the pith is recovered, representing about 80% by weight of the fresh cane. Approximately 150 kg of the pith are placed in a 1000 L vessel together with 400 L of tap water. The pH is adjusted to a value of 4.0-4.5 with a 10 N solution of HCl and the whole mixture is cooled and inoculated with a 24-h old inoculum of a strain of Saccharomyces cerevisae (yeast), viz. CBS 2959 strain. The volume of inoculum is 80 L, with about 15 g/L of total sugars, about 28 g/L of ethanol and about 9 g/L of dry biomass. The vessel is incubated at 30° C. for 40 h without agitation. An ethanolic-yeast suspension is obtained. Solid cane pieces or particles are thereafter separated from the ethanolic-yeast suspension by a filter. The resulting filtrate has an ethanol concentration of about 27 g/L, a total sugars concentration of about 0.2 g/L and a dry biomass concentration of about 3 g/L. About 14.4 kg of ethanol are produced. The consumption of extractable sugars is above 99%; the ethanol yield is about 0.60 kg ethanol/kg sucrose consumed; the total yeast biomass produced is about 1.2 kg and the yeast yield is about 0.05 kg dry biomass/kg sucrose consumed. About 340 L of this ethanolic-yeast suspension are then mixed with about 30 kg of cane pith previously dried in a forced-air tray dryer employing air at 60° C. to reach a final moisture of about 2%. The vessel is incubated at 30° C. for 24 h. After that time, an ethanol-yeast suspension is again separated by filtration. The composition of the resulting filtrate is about 50 g/L of ethanol, about 0.6 g/L of total sugars and about 6 g/L of dry biomass. The ethanol produced in this second fermentation is about 9.4 kg; more than 98% of the total extractable sugars are consumed; the yield is about 0.63 kg ethanol/kg sucrose consumed; the total yeast biomass produced is about 850 g and the yeast yield is about 0.06 kg dry biomass/kg sucrose consumed. In all, the yield is about 60 kg bioethanol/100 kg sucrose.

### Conversion of Benzyl Alcohol and Benzaldehyde into Ethanol, in Accordance with Step (B) of the Invented Process

### Catalytic Reaction at 325° C.

Catalytic reaction is carried out in a packed-bed reactor. The reaction temperature is maintained using a vertically aligned tube furnace equipped with a thermocouple.

150 g of 0.8 wt % Co/HAP catalyst are pre-treated at 400° C. for 2 h in 8% H₂/N₂ prior to the reaction. The reaction is carried out under atmospheric pressure with a total gas flow rate of 30 L/min. For the whole reaction, the feed gas is 4 vol % methanol and 2 vol % ethanol, and is balanced with nitrogen. The weight hourly space velocities of ethanol and methanol are 0.37 g ethanol/_{(g catalyst·h)} and 0.51 g methanol/_{(g catalyst·h)}, respectively. The reaction temperature is set at 325° C. The reaction is operated for several tens of hours at 325° C., during which the Co/HAP exhibits a good stability.

To determine the composition of the organic products exiting the packed bed reactor, these ones are collected in cold traps with acetone and analyzed using an on-line gas chromatograph (GC) equipped with a flame ionization detector. The residence time for each specific component is determined using the corresponding standard chemicals. The identities of the organic products are further confirmed by GC/MS analysis. The organic products include benzaldehyde, benzyl alcohol, unconverted methanol, unconverted ethanol and various by-products, viz. propanol, butanol, pentanol, hexanol, 2-methylbenzyl alcohol, 4-methylbenzyl alcohol, acrolein, acetaldehyde, E-2-butenal, 2-tolualdehyde and 4-tolualdehyde.

Under the above operating conditions, the ethanol conversion achieved after one pass in the reactor ranges from about 38% to about 47%. The selectivity towards the benzyl alcohol is about 22% and the selectivity towards the benzaldehyde is about 4%.

The benzyl alcohol and the benzaldehyde are separated from the unconverted ethanol, the unconverted methanol and the reaction by-products (butanol, pentanol, hexanol, 2-methylbenzyl alcohol, 4-methylbenzyl alcohol, acrolein, acetaldehyde, E-2-butenal, 2-tolualdehyde and 4-tolualdehyde) by distillation. To get rid of the 2-methylbenzyl alcohol, the 4-methylbenzyl alcohol, the 2-tolualdehyde and the 4-tolualdehyde, a distillation under vacuum is operated with a pot temperature of 120-160° C.

The unconverted ethanol and the unconverted methanol are recycled, that is to say they are re-engaged in a new catalytic reaction, in admixture with fresh methanol and ethanol originating from the step (A); the amounts of fresh methanol and ethanol are such that they compensate the respective amounts of methanol and ethanol that have been converted during the first catalytic reaction. The new catalytic reaction is carried out using the same equipment and the same operating conditions as above detailed. The newly obtained benzyl alcohol and benzyl alcohol are again separated from the unconverted ethanol, the unconverted methanol and the reaction by-products by distillation. After this second pass, the recycling of the still unconverted ethanol in the reactor followed by the separation of the reaction products may then be repeated as much as needed to reach finally an ethanol conversion which approaches 100%. By doing so, the combined yield in benzyl alcohol and in benzaldehyde reaches about 250 g/kg ethanol.

All in all, about 2.8 kg of ethanol produced during the step (A) are converted, and about 700 g of benzyl alcohol and benzaldehyde are recovered.

### Catalytic Reaction at 350° C.

The catalytic reaction is carried out using the same equipment and the same operating conditions as above detailed, except that the reaction temperature is set to 350° C. The selectivity towards the benzyl alcohol is about 24% and the selectivity towards the benzaldehyde is about 8%. After recycling so as to reach a final ethanol conversion approaching 100%, the combined yield in benzyl alcohol and in benzaldehyde slightly exceeds 300 g/kg ethanol.

### Catalytic Reaction at 375° C.

The catalytic reaction is carried out using the same equipment and the same operating conditions as above detailed, except that the reaction temperature is set to 375° C. The selectivity towards the benzyl alcohol is about 21% and the selectivity towards the benzaldehyde is about 14%. After recycling so as to reach a final ethanol conversion approaching 100%, the combined yield in benzyl alcohol and in benzaldehyde is about 350 g/kg ethanol.

### Oxidation of Benzyl Alcohol and Benzaldehyde to Benzoic Acid, in Accordance with Step (C) of the Invented Process

4.2 g (0.5 wt % catalyst loading) of 5 wt % Pt-1.5 wt % Bi/C (“160 paste”, moisture 46.8%), 500 g of a mixture of benzaldehyde and benzyl alcohol from the step (B), 1 kg of KOH, 500 mL of methanol, and 4.5 L of pure H₂O are added sequentially into a 10 L reactor. The reactor is then closed and charged with 1 bar of oxygen. Next, the reactor is heated at 50° C. The reaction pressure of O₂ is kept at 1 bar during the whole process. Reaction samples are taken from the reactor and analyzed by GC so as to check if essentially the whole amount of the benzaldehyde and the benzyl alcohol is fully converted. When it is so, the reaction mixture is acidified with 1 N HCl aqueous solution, which is all filtered and transferred in a storage vessel. The yield for benzoic acid is determined by 1H NMR analysis using 1,3,5-trimethoxybenzene as the internal standard and CD₃OD as deuterated solvent. For the isolation of benzoic acid, after the acidification, the catalyst is separated by centrifugation and the liquid part is extracted by ethyl acetate to obtain the organic solution of benzoic acid. The latter is then distilled under vacuum to obtain 490 g of benzoic acid with a purity of higher than 99% at a temperature of 170-200° C. In all, the yield is 98 g benzoic acid/100 g mixture of benzaldehyde and benzyl alcohol.

### Oxidation of Benzoic Acid to Phenol, in Accordance with Step (D) of the Invented Process

The reaction is carried out using a fixed-bed reactor at atmospheric pressure. The reactor is made of a quartz tube with an inner diameter of 20 mm and a length of 500 mm. The experiments are carried out in the presence of steam. 400 g of molten benzoic acid obtained from the step (C) is supplied to the reactor with a hot steal syringe which is heated to 130° C. An amount of 1.5 g of the NiO—Fe₂O₃—Na₂O—V₂O₅ catalyst catalysts is used and the reaction temperature is measured by a thermocouple placed in a thermowell within the catalyst bed. The reaction temperature is set at 400° C. The inlet flow consists of benzoic acid, air and steam in a weight ratio of 1:5.5:21 at a space velocity of 1500 h⁻. The organic products are collected in cold traps with acetone and analyzed on-line using a GC equipped with a FID Detector. Phenol, benzene, carbon monoxide, carbon dioxide and trace amounts of biphenyl and phenyl benzoate are detected in the analysis. The selectivities to the products are calculated on the basis of the benzoic acid which is converted. The NiO—Fe₂O₃—Na₂O—V₂O₅ catalyst proves to exhibit a good stability. The conversion of benzoic acid is about 95% and the selectivity to phenol is about 90%. For the isolation of phenol, the reaction products are distilled under vacuum at a temperature of 100-130° C.; more than 250 g phenol with a purity of higher than 99% are recovered.

1. A process for the production of phenol, said process comprising the steps of:
(A) producing ethanol from a biomass, then
(B) causing the ethanol produced at the step (A) to react with methanol, so as to produce benzyl alcohol and/or benzaldehyde, then
(C) causing the benzyl alcohol and/or benzaldehyde produced at the step (B) to react with oxygen, so as to produce benzoic acid, then
(D) causing the benzoic acid produced at the step (C) to react with oxygen, so as to produce phenol.

2. The process according to claim 1, wherein the biomass contains a carbohydrate, wherein the carbohydrate is a monosaccharide.

3. (canceled)

4. The process according to claim 1, wherein the reaction of the step (A) is a fermentation reaction and the fermentation takes place in a fermentation medium comprising a microorganism, wherein the microorganism is a yeast selected from the group consisting of Saccharomyces spp. and Brettanomyces custersii.

5. (canceled)

6. (canceled)

7. The process according to claim 1, wherein the step (B) takes place in the presence of a transition metal catalyst supported on a support, wherein the transition metal is chosen from cobalt, nickel, copper, silver, iridium, zinc and yttrium, and wherein the support is hydroxyapatite of formula Ca₁₀(OH)₂(PO₄)₆.

8. (canceled)

9. (canceled)

10. (canceled)

11. (canceled)

12. (canceled)

13. The process according to claim 1, wherein the step (C) takes place in the presence of a transition metal catalyst and, optionally in addition, of a co-catalyst, wherein, the catalyst and, when present, the co-catalyst are supported on a same support.

14. The process according to claim 13, where the transition metal is a noble metal, wherein the noble metal is platinum.

15. (canceled)

16. The process according to claim 13, wherein the co-catalyst is present and is a poor metal, wherein the poor metal is bismuth.

17. (canceled)

18. (canceled)

19. The process according to claim 1, wherein the step (D) takes place in the presence of a catalyst, said catalyst comprising an oxide of a transition metal and said transition metal catalyst being an element chosen from the elements of groups 8 to 10 of the periodic table optionally in combination with an element chosen from the elements of groups 4 to 7.

20. The process according to claim 19, wherein the catalyst further comprises an oxide of an alkali or alkaline earth metal, wherein the catalyst consists essentially of or consists of a mixture of Fe₂O₃, NiO, V₂O₅ and Na₂O.

21. (canceled)

22. (canceled)

23. The process according to claim 1, wherein:
the step (A) comprises (A-1) causing a biomass to undergo a chemical transformation in a first reaction medium, so as to produce ethanol in the first reaction medium, then (A-2) separating the ethanol from the first reaction medium;
the step (B) comprises (B-1) causing the so-separated ethanol to react with methanol in a second reaction medium, so as to produce benzyl alcohol and/or benzaldehyde in the second reaction medium, then (B-2) separating the benzyl alcohol and/or benzaldehyde from the second reaction medium;
the step (C) comprises (C-1) causing the so-separated benzyl alcohol and/or benzaldehyde to react with oxygen in a third reaction medium, so as to produce benzoic acid in the third reaction medium, then (C-2) separating the benzoic acid from the third reaction medium; and
the step (D) comprises (D-1) causing the so-separated benzoic acid to react with oxygen in a fourth reaction medium, so as to produce phenol in the fourth reaction medium, then (D-2) separating the phenol from the fourth reaction medium.

24. The process according to claim 23, wherein the sub-step (B-2) further comprises separating the unconverted ethanol from the second reaction medium and recycling it one or several times, that is to say re-using it as reactant of a new second reaction medium in accordance with the sub-step (B-1).

25. The process according to claim 23, wherein the sub-step (B-2) further comprises separating the unconverted methanol from the second reaction medium and recycling it one or several times, that is to say re-using it as reactant of a new second reaction medium in accordance with the sub-step (B-1).

26. Use of 20 parts by weight or less of a carbohydrate contained in a biomass for the production of 1 part of phenol, wherein the carbohydrate is a monosaccharide, and wherein the biomass is a sugarcane.

27. (canceled)

28. (canceled)

29. (canceled)

30. Use according to claim 26, wherein 10 parts by weight or less of the carbohydrate contained in the biomass are used for the production of 1 part of phenol.

31. A method for the synthesis of a bisphenol compound, which comprises:
producing phenol by the process according to claim 1, then
causing the so-produced phenol to be condensed with an aldehyde or a ketone, or to be sulfonated and condensed with sulfuric acid or sulfur trioxide.

32. The method according to claim 31, wherein the phenol is condensed with acetone and the bisphenol compound is bisphenol A, and wherein the phenol is sulfonated and condensed with sulfuric acid or sulfur trioxide and the bisphenol compound is bisphenol S.

33. (canceled)
